(19) United States
(12) Patent Application Publication
(10) Pub. No.: US 2024/0399023 A1
(43) Pub. Date: Dec. 5, 2024

(54) Polydioxanone Composite Coated Magnesium Calcium Phosphate Composite Alloy for Orthopedic Implants

(71) Applicant: Robert A. Flavin, Dublin (IE)

(72) Inventor: Robert A. Flavin, Dublin (IE)

(21) Appl. No.: 18/672,079

(22) Filed: May 23, 2024

Related U.S. Application Data
(60) Provisional application No. 63/470,192, filed on Jun. 1, 2023.

(51) Int. Cl. A61L 27/04; A61L 27/12; A61L 27/34; A61L 27/58; C08L 67/04

(52) U.S. Cl. CPC A61L 27/047; A61L 27/12; A61L 27/34; A61L 27/58; C08L 67/04; A61L 2430/02

(57) ABSTRACT

A polymer composite alloy for orthopedic implants includes a magnesium alloy coated with a polymer composite. The polymer composite can be polydioxanone. Alternatively, the polymer composite can be polyglyconate. Implants manufactured from the alloy composite are also provided. In an exemplary embodiment, the implant includes at least one through passage from one side of the implant to a distal side of implant.

### BACKGROUND OF THE INVENTION

### Field of the Invention

The invention relates to a polydioxanone (“PDS”) composite coated magnesium calcium phosphate composite alloy for orthopedic implants.

### Description of the Related Art

Titanium and 316L stainless steel have been the mainstay metals for orthopedic implants for over a 100 years. Nickel allergies and seeking more biocompatible implants has led to titanium superseding 316L SS. One drawback is that both materials have no dynamic capabilities which leads to stress shielding and the potential to cause bones to not heal. Also the increasing trend to removal of orthopedic implants has led to a huge burden on the orthopedic resources in many countries.

It would be beneficial to develop a PDS composite coated magnesium alloy implant to: aid in stability of any bone healing environment; dynamically change with the environment to allow the bone healing environment to exhibit more load as the bone heals; ultimately absorb into the surrounding bone tissue to avoid removal; produce an osteoblastic environment which promotes bone healing rather then cause a static environment; and reduce the incidence of a burst or rebound Osteoclastic event by controlling the elution of Mg into the environment.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention provides a polydioxanone composite coated magnesium calcium phosphate composite alloy for orthopedic implants. Implants manufactured from the alloy composite are also provided.

In another embodiment, the present invention provides a polyglyconate (co-polymer of Glycolic Acid & Trimethylene Carbonate) composite coated magnesium calcium phosphate composite alloy for orthopedic implants. Implants manufactured from the alloy composite are also provided.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a top plan view of an exemplary implant coated with the inventive composite and having a through passage for bone growth; and

FIG. 2 is a sectional view of the implant of FIG. 1 taken through lines 2-2 of FIG. 1.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

The present invention provides a polydioxanone ((C₄H₆O₃)ₙ) composite coated magnesium calcium phosphate composite alloy for orthopedic implants that includes a magnesium alloy coated with a polydioxanone composite.

In an exemplary embodiment, the magnesium alloy comprises or alternatively consists essentially of:
magnesium 95-99% by weight;
octacalcium phosphate (OCP) 0.1-4% by weight;
dicalcium phosphate dihydrate (DCPD) 0.1-4% by weight;
zinc 0.1-3% by weight;
manganese 0.1-3% by weight; and
vitamin D 0.1-4% by weight,
with the possible existence of trace impurities in the alloy.

The polydioxanone composite also includes an adhesive to adhere the polydioxanone composite to the magnesium alloy. Exemplary adhesives can be fibrin, phospherine, and dopamine. In an exemplary embodiment, the polydioxanone composite coating is between about 0.05 mm and about 1.5 mm thick. The inventor has found that layers greater than 1.5 mm thick have a tendency to peel off the magnesium alloy base composite material.

In an alternative embodiment, a glycolide/trimetheylene carbonate (polyglyconate) polymer coating can be applied to the magnesium alloy.

In an exemplary embodiment, the alloy forms an implant, such as a wire, an intermedullary nail, a fixation nail, an osteotomy a wedge, a screw, and a plate. Exemplary wedges are foot & ankle wedges, such as a Cotton Wedge, an Evans Wedge, and a distal tibial osteotomy (DTO) wedge. Other exemplary wedges are a knee wedge, a distal femoral wedge, and a high tibial osteotomy (HTO) wedge. All implants have rounded off edges.

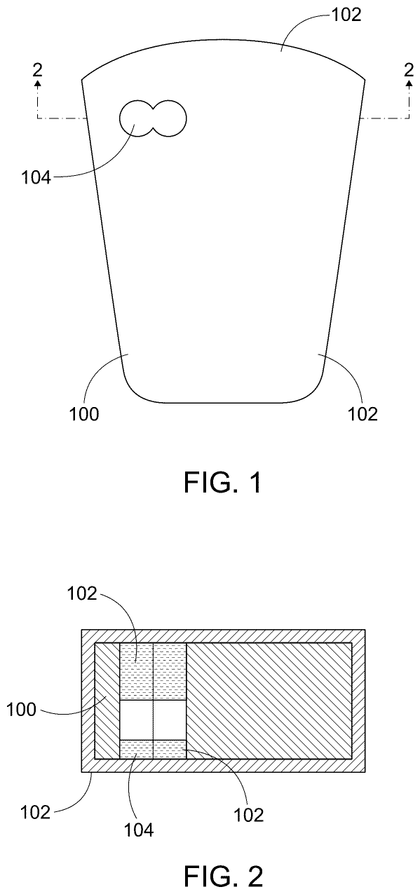

Referring to FIGS. 1 and 2, a wedge 100 according to an exemplary embodiment of the invention is coated with polydioxanone and/or a polyglycnate coating 102 and includes at least one through passage 104 from one side of implant 100 to a distal side of implant 100. In an exemplary embodiment, at least one through passage 104 is shaped like the infinity sign in its cross section and is designed to allow bone to grow from one side of implant 100 to the other. Although only one through-passage 104 is shown in the Figures, those skilled in the art will recognize that more than one through-passage 104 can be provided in implant 100, and through-passages 104 can extend along different axes.

The through passages 104 are 50-90% covered by the polydioxanone/polyglyconate composite so the magnesium alloy becomes exposed quicker to allow bone expansion. The infinity sign shape of through passages 104 enables double pillar bone growth, which aids in stronger bone struts with the initial healing phase. These pillar structures expand as the magnesium is absorbed by the body to allow for bone growth. The magnesium alloy has OCP and DCPD mixed in, which promotes bone growth as the alloy absorbs, while delaying its absorption by the body.

It will be further understood that various changes in the details, materials, and arrangements of the parts which have been described and illustrated in order to explain the nature of this invention may be made by those skilled in the art without departing from the scope of the invention as expressed in the following claims.

I claim:

1. A polymer composite alloy for orthopedic implants comprises:
a magnesium alloy; and
a polymer composite coating the magnesium alloy.

2. The polymer composite alloy according to claim 1, wherein the polymer comprises polydioxanone.

3. The polymer composite alloy according to claim 1, wherein the magnesium alloy comprises:
magnesium 95-99% by weight;
octacalcium phosphate 0.1-4% by weight;
dicalcium phosphate dihydrate 0.1-4% by weight;
zinc 0.1-3% by weight;
manganese 0.1-3% by weight; and
vitamin D 0.1-4% by weight.

4. The polymer composite alloy according to claim 1, wherein the composite comprises an adhesive.

5. The polymer composite alloy according to claim 4, wherein the adhesive is at least one from the group consisting of fibrin, phospherine, and dopamine.

6. The polymer composite alloy according to claim 1, wherein the alloy forms an implant.

7. The polymer composite alloy according to claim 6, wherein the implant is one from the group consisting of a wire, an intermedullary nail, a fixation nail, wedge, a screw, and a plate.

8. The polymer composite alloy according to claim 7, wherein, when the implant is a wedge, the wedge having a through passage extending therethrough.

9. The polymer composite alloy according to claim 8, wherein the through passage has an infinity shaped cross section.

10. The polymer composite alloy according to claim 8, wherein the through passage is 50-90% covered by the polymer composite.

11. The polymer composite alloy according to claim 1, wherein the polymer composite is between about 0.05 mm and about 1.5 mm thick.

12. The polymer composite alloy according to claim 1, wherein the polymer comprises polyglyconate.

13. The polymer composite alloy according to claim 1, wherein the magnesium alloy consists essentially of:
magnesium 95-99% by weight;
octacalcium phosphate 0.1-4% by weight;
dicalcium phosphate dihydrate 0.1-4% by weight;
zinc 0.1-3% by weight;
manganese 0.1-3% by weight; and
vitamin D 0.1-4% by weight.

14. A polymer composite alloy comprising:
a magnesium alloy consisting essentially of:
magnesium 95-99% by weight;
octacalcium phosphate 0.1-4% by weight;
dicalcium phosphate dihydrate 0.1-4% by weight;
zinc 0.1-3% by weight;
manganese 0.1-3% by weight; and
vitamin D 0.1-4% by weight; and
a polymer composite coating.

15. The polymer composite alloy according to claim 14, wherein the polymer composite coating comprises polydiaxonone.

16. The polymer composite alloy according to claim 14, wherein the polymer composite coating comprises polyglyconate.

17. A polymer composite alloy comprising:
a magnesium alloy comprising:
magnesium 95-99% by weight;
octacalcium phosphate 0.1-4% by weight;
dicalcium phosphate dihydrate 0.1-4% by weight;
zinc 0.1-3% by weight;
manganese 0.1-3% by weight; and
vitamin D 0.1-4% by weight; and
a polymer composite coating.

18. The polymer composite alloy according to claim 17, wherein the polymer composite coating comprises polydiaxonone.

19. The polymer composite alloy according to claim 17, wherein the polymer composite coating comprises polyglyconate.
